(19) United States
(12) Patent Application Publication
(10) Pub. No.: US 2020/0408761 A1
(43) Pub. Date: Dec. 31, 2020

(54) METHODS OF DIAGNOSING AND TREATING BLADDER CANCER

(71) Applicant: The Medical Research, Infrastructure and Health Services Fund of the Tel Aviv Medical Center, Tel-Aviv (IL)

(72) Inventors: Nadir ARBER, Tel-Aviv (IL); Shiran SHAPIRA, Petach-Tikva (IL); Dina KAZANOV, Rishon-LeZion (IL)

(73) Assignee: The Medical Research, Infrastructure and Health Services Fund of the Tel Aviv Medical Center, Tel-Aviv (IL)

(21) Appl. No.: 16/976,143

(22) PCT Filed: Feb. 28, 2019
(86) PCT No.: PCT/IL2019/050227

§ 371 Date: Aug. 27, 2020

Related U.S. Application Data
(60) Provisional application No. 62/636,224, filed on Feb. 28, 2018.

(51) Int. Cl. G01N 33/574

(52) U.S. Cl. CPC G01N 33/57407; G01N 2333/70596

(57) ABSTRACT

A method of diagnosing high grade bladder cancer is provided. The method comprising detecting in a urine sample of a subject in need thereof expression of CD24, wherein an increase in said expression of CD24 above a predetermined threshold as compared to a control sample is indicative of said high grade bladder cancer.

### RELATED APPLICATION/S

This application claims the benefit of priority of U.S. Provisional Patent Application No. 62/636,224 filed on Feb. 28, 2018, the contents of which are incorporated herein by reference in their entirety.

### FIELD AND BACKGROUND OF THE INVENTION

The present invention, in some embodiments thereof, relates to methods of diagnosing and treating bladder cancer.

Bladder cancer (BC) is a relatively common and strikingly costly malignancy associated with high recurrence and mortality rates (Siegel et al., 2015). It is the fifth most commonly diagnosed cancer in the U.S. and the 4^{th} most common cause of death due to cancer.

Urothelial carcinoma (UC) is the main BC subtype accounting for 90% of all cases (Rahmani et al., 2013). At the time of diagnosis about 20% of UCs have invaded the muscle layer of the bladder wall. Such cancer is associated with poor prognosis. In addition, papillary and superficial tumors recur in 70% of patients after surgical excision of the tumor (Goodison et al., 2013). Therefore, development of accurate surveillance tests in addition to cystoscopy to evaluate disease aggressiveness and prognosis is still a major clinical need.

Currently, cystoscopy and urine cytology are the recommended means for BC diagnosis (Griffiths, 2013). In fact, direct cystoscopic visualization of the bladder is the gold standard diagnostic assessment. Newer technologies including tomography and confocal laser endomicroscopy may improve the sensitivity and specificity of identifying BCs however, although these methods have a high detection rate, they are expensive and invasive. Urine cytology is a non-invasive method for detecting BC by identifying abnormal urothelial cells. It has high specificity but relatively low sensitivity, particularly in well-differentiated low grade bladder tumors (Ye at al., 2014).

Urinary markers seem to be promising tools for diagnosis and follow-up of BC. Many soluble markers have been explored for BC diagnosis and screening. Several of these markers, including bladder tumor antigen (BTA-stat, BTA-TRAK), nuclear matrix protein-22 and fibrinogen degradation products, have been approved by the FDA for clinical use. Such proteins can be detected in urine and used in conjunction with cystoscopy to facilitate diagnosis and monitoring of BC patients. However, these tests have high false positive rates due to the presence of inflammatory cells and other contaminating cells (Ye at al., 2014).

CD24, a mucin-like cell surface molecule and P-selectin ligand, is a hematopoietic receptor that has also been identified as a stem cell marker but no function has yet been ascribed (Fillmore et al., 2007). The CD24 gene encodes a cell surface molecule, which is a heavily glycosylated phosphatidylinositol-anchored mucin-like protein (Kristiansen et al., 2004). Physiologically, CD24 is expressed mainly on premature lymphocytes and epithelial cells (reviewed in Sagiv & Arber, 2008). Its expression is also reported during the embryonic period, on developing neural cells (Poncet et al., 1996). CD24 was shown to be overexpressed in a variety of malignancies including B-cell lymphomas, gliomas, small-cell and non-small cell lung, hepatocellular, renal cell, nasopharyngeal, uterine, epithelial ovarian, breast, prostate, pancreatic, colorectal, and bladder carcinomas (reviewed in Sagiv & Arber, 2008). Increased expression of CD24 is usually tied with a more aggressive course of the disease. CD24 protein functions as an adhesion molecule and therefore, it might as well enhance metastases shedding, which may explain its association with a worse prognosis (Lee et al., 2009).

In the context of BC, the expression of CD24 has been evaluated in a tissue microarray and shown to be an independent prognostic factor (Smith et al., 2006). Thus, multivariate analysis showed that increased expression of CD24 correlated with shorter patient disease-free survival thereby suggesting CD24 as a potential prognostic marker for BC. In vitro, tumor cell CD24 expression correlated with a propensity to metastasize to the lung in a murine model of human metastatic BC (Overdevest et al., 2011). Suppression of CD24 reduced acute tumor cell retention in the lungs of mice inoculated with cancer cells. Moreover, immunohistochemical (IHC) evaluation of paired primary and metastatic human BC samples revealed increased staining intensity and frequency of CD24 expression in metastases (Overdevest et al., 2011). Stratification of patients based on IHC expression in their tumors revealed that high levels of CD24 are associated with poorer prognosis in males. Thus, CD24 seems to play a significant role in bladder tumorigenesis and metastasis, particularly in males (Overdevest et al., 2012). Recently, CD24 expression in cancer tissues obtained during transurethral surgery and the subsequent intra-bladder tumor recurrence were assessed (Liu et al., 2013). CD24 expression was observed more frequently in high-grade than low-grade bladder tumors. Positive CD24 expression was significantly associated with intra-bladder recurrence following surgery and increased staining intensity was also correlated with recurrence, suggesting that the expression of CD24 is significantly associated with BC recurrence following treatment, may serve as a predictive marker, and a potential target for treatment.

PCT Publication No. WO2007/088537 teaches the use of CD24 as a marker for early diagnosis of bladder cancer.

PCT Publication No. WO2009/074988 teaches the use of CD24 on lymphocytes as a marker for diagnosis of cancer.

### SUMMARY OF THE INVENTION

According to an aspect of some embodiments of the present invention there is provided a method of diagnosing high grade bladder cancer, the method comprising detecting in a urine sample of a subject in need thereof expression of CD24, wherein an increase in the expression of CD24 above a predetermined threshold as compared to a control sample is indicative of the high grade bladder cancer.

According to an aspect of some embodiments of the present invention there is provided a method of treating bladder cancer in a subject in need thereof, the method comprising:

(a) obtaining a urine sample from the subject;

(b) detecting in the urine sample of the subject expression of CD24;

(c) diagnosing the subject with high grade bladder cancer when the expression of CD24 above a predetermined threshold as compared to a control sample is detected; and

(d) administering to the subject an effective amount of a high grade bladder cancer therapy.

According to some embodiments of the invention, the subject is diagnosed with bladder cancer.

According to an aspect of some embodiments of the present invention there is provided a method of monitoring treatment of high grade bladder cancer, the method comprising:

(a) treating a subject having high grade bladder cancer with a high grade bladder cancer therapy; and

(b) detecting in the urine sample of the subject expression of CD24, wherein a decrease in the expression of CD24 below a predetermined threshold as compared to expression of same prior to treatment is indicative of the therapy being efficacious.

According to an aspect of some embodiments of the present invention there is provided a method of detecting CD24 is a subject, the method comprising:

(a) obtaining a urine sample from the subject;

(b) processing the urine sample so as to obtain a urine sample comprising exosomes or non-cellular particles in an amount not exceeding 5×10⁷/ml; and

(c) detecting expression of CD24 in the processed urine sample.

According to an aspect of some embodiments of the present invention there is provided a composition of matter comprising a urine sample of a subject diagnosed with bladder cancer, and an agent capable of detecting CD24.

According to some embodiments of the invention, the bladder cancer is high grade bladder cancer.

According to some embodiments of the invention, the urine sample comprises exosomes or non-cellular particles in an amount not exceeding 5×10⁷/ml.

According to some embodiments of the invention, the urine sample comprises intact cells in an amount not exceeding 10 cells/ml.

According to some embodiments of the invention, the urine sample is less than 6 hours.

According to some embodiments of the invention, the CD24 comprises CD24 polypeptide.

According to some embodiments of the invention, the CD24 comprises CD24 mRNA.

According to some embodiments of the invention, the detecting is by using an immunoassay using an antibody.

According to some embodiments of the invention, the immunoassay comprises an ELISA assay.

According to some embodiments of the invention, the agent is an antibody.

According to some embodiments of the invention, the composition comprising a secondary antibody capable of binding said antibody.

According to some embodiments of the invention, the antibody comprises SWA11.

According to some embodiments of the invention, the agent is an oligonucleotide.

According to some embodiments of the invention, the method further comprises corroborating the diagnosis using a Gold standard assay.

According to some embodiments of the invention, the Gold standard assay is selected from the group consisting of cystoscopy, TURP and ureteroscopy.

According to some embodiments of the invention, the control sample is of a healthy subject, a subject not having a malignancy of the bladder or a subject having a low grade bladder cancer.

According to some embodiments of the invention, the control sample is of a healthy subject or a subject having a low grade bladder cancer.

According to some embodiments of the invention, the processing comprises centrifugation under a centrifugal force not exceeding 2000×g.

According to some embodiments of the invention, the processing does not comprise ultracentrifugation.

According to some embodiments of the invention, the expression of the CD24 is detected as a single marker.

According to some embodiments of the invention, the method comprises detecting expression of markers distinct from the CD24, wherein the markers do not exceed 3 distinct markers.

According to some embodiments of the invention, the composition does not comprise an agent capable of detecting markers distinct from the CD24.

According to some embodiments of the invention, the composition comprises an agent capable of detecting markers distinct from the CD24, wherein the markers do not exceed 3 distinct markers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing the concentration of CD24 in urine samples of high and low grade BC compared to urine samples of healthy subjects or urologic patients without malignancy of the bladder. The amount of CD24 in urine samples was determined by sandwich ELISA using anti-CD24 capture and detecting antibodies. The results in the bar graph represent the average values±SD. The amount of CD24 was calculated based on a standard curve prepared using a purified recombinant CD24 protein. Normal—healthy subjects or urologic patients without malignancy of the bladder; Low—patients with low grade tumors; High—patients with high grade tumors.

FIGS. 2A and 2B are bar graphs showing the optical density (OD) values of CD24 in urine samples of BC patients compared to urine samples of healthy subject. CD24 in urine samples was evaluated by sandwich ELISA using anti-CD24 capture and detecting antibodies. The results in the bar graphs represent the average values±SD. The relative amount of CD24 in the different groups can be determined based on the OD values at 450 nm. N—healthy subjects or urologic patients without malignancy of the bladder; LG—patients with low grade tumors; HG—patients with high grade tumors.

FIG. 3 is a bar graph showing CD24 levels in urine samples of high and low grade BC relative to the levels in healthy subjects or urologic patients without malignancy of the bladder. The amount of CD24 in urine samples was determined by sandwich ELISA using anti-CD24 capture and detecting antibodies. The results in the bar graph represent the average values±SD, n=92. The amount of CD24 was calculated based on a standard curve prepared using a purified recombinant CD24 protein and is presented as fold change compared to the level determined in the urine of healthy subjects or urologic patients without malignancy of the bladder. LG—patients with low grade tumors; HG—patients with high grade tumors.

### DESCRIPTION OF SPECIFIC EMBODIMENTS OF THE INVENTION

The present invention, in some embodiments thereof, relates to methods of diagnosing and treating bladder cancer.

Bladder cancer (BC) is a relatively common and strikingly costly malignancy associated with high recurrence and mortality rates (Siegel et al., 2015).

Currently, cystoscopy and urine cytology are the recommended means for BC diagnosis.

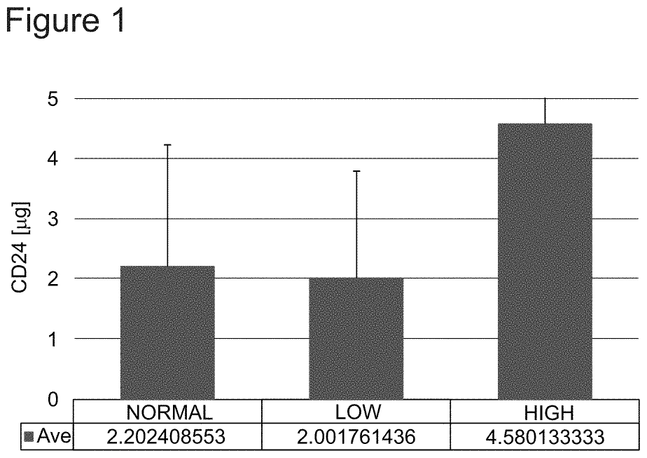

While reducing the present invention for practice, the present inventors have now discovered that CD24 levels in urine samples obtained from patients having high grade bladder cancer were higher compared to CD24 levels in urine samples obtained from healthy subjects or urologic patients without malignancy of the bladder; and also compared to CD24 levels in urine samples from patients having low grade bladder cancer (Example 1, FIGS. 1-3, in the Examples section which follows).

Consequently, specific embodiments of the present invention suggest the use of CD24 levels in urine samples for diagnosing, staging and treating bladder cancer.

Thus, according to a first aspect of the present invention, there is provided a method of diagnosing high grade bladder cancer, the method comprising detecting in a urine sample of a subject in need thereof expression of CD24, wherein an increase in said expression of CD24 above a predetermined threshold as compared to a control sample is indicative of said high grade bladder cancer.

As used herein the term “urothelial cancer” or “UC” or “bladder cancer” or “transitional cell carcinoma (TCC)”, refers to the most common type of bladder cancer. This type of cancer typically starts in the urothelial cells that line the inside of the bladder. Urothelial cells also line other parts of the urinary tract, such as the part of the kidney that connects to the ureter (called the renal pelvis), the ureters, and the urethra.

UC is often described based on invasiveness. Non-invasive cancers are still in the inner layer of cells (the transitional epithelium) but have not grown into the deeper layers. Invasive cancers have grown into deeper layers of the bladder wall. UC can also be classified as superficial or non-muscle invasive. These terms include both non-invasive tumors as well as any invasive tumors that have not grown into the main muscle layer of the bladder.

UCs are also divided into 2 subtypes, papillary and flat. Papillary carcinomas grow in slender, finger-like projections from the inner surface of the bladder toward the hollow center. Papillary tumors often grow toward the center of the bladder without growing into the deeper bladder layers. These tumors are called noninvasive papillary cancers. Very low-grade (slow growing), non-invasive papillary cancer is sometimes called papillary urothelial neoplasm of low-malignant potential (PUNLMP) and tends to have a very good outcome. Flat carcinomas do not grow toward the hollow part of the bladder at all. If a flat tumor is only in the inner layer of bladder cells, it is known as a non-invasive flat carcinoma or a flat carcinoma in situ (CIS). If either a papillary or flat tumor grows into deeper layers of the bladder, it is called an invasive urothelial (or transitional cell) carcinoma.

According to the American Joint Committee on Cancer/International Union Against Cancer/Union Internationale Contre le Cancer (AJCC/UICC) TNM system[20], UC is staged as follows:

Tumor is characterized at its primary niche (T, wherein Tx stands for inability to assess primary tumor; T0—negation of tumor evidence; Ta—noninvasive papillary carcinoma; Tis—carcinoma in situ; T1—tumor invades sub-epithelial connective tissue; T2—muscle invasion; T3—invasion to perivesical tissues; T4a—invasion to either prostate stroma, seminal vesicles, uterus or vagina, and T4b—metastasis to pelvic wall or abdominal wall) and may also have metastases in regional lymph nodes (N, wherein Nx stands for inability to assess nodes; N0—negation of regional node metastases; N1 & N2—one to multiple metastatic nodes, respectively and N3—metastasis in the common iliac lymph nodes) and in distant location (M1, as opposed to M0—no distant metastases). Based on the elaborated above TMN system, the overall staging definition may range from stages 0a and 0is (Ta and Tis, respectively, N0 and M0) via stages I to III (T1 to T4a, N0 and M0), to stage IV (with three sub-stages: T4b,N0,M0; any T,N1-3,M0; any T or N,M1).

As used herein the term “subject” refers to a human subject who is at risk of having UC [e.g., a genetically predisposed subject, a subject with medical and/or family history of cancer, a subject who has been exposed to carcinogens, occupational hazard, environmental hazard] and/or a subject who exhibits suspicious clinical signs of UC (as further described hereinbelow). Additionally or alternatively, the subject in need thereof can be a healthy human subject undergoing a routine well-being check-up.

According to specific embodiments, the subject is diagnosed with UC.

The subject may be of any age, gender and ethnic group with the following notes. UC typically occurs in older people. About 9 out of 10 people with this cancer are over the age of 55. The average age at the time of diagnosis is 73. Men are about 3 to 4 times more likely to get UC during their lifetime than women. White Caucasians are diagnosed with UC about twice as often as African Americans or Hispanic Americans.

Thus, according to specific embodiments, the subject is a male.

According to specific embodiments, the subject is over the age of 50.

Signs and symptoms of UC include blood in the urine and changes in urination, as further explained hereinbelow.

Blood in the urine—In most cases, blood in the urine (hematuria) is the first sign of UC. Usually, the early stages of UC cause bleeding but little or no pain or other symptoms.

Changes in bladder habits or symptoms of irritation—UC can sometimes cause changes in urination, such as: having to urinate more often than usual; pain or burning sensation during urination.

Risks for UC include, but are not limited to, smoking, exposure to aromatic amines, use of medicines and herbal supplements of specific groups e.g., pioglitazone (Actos), arsenic in drinking water, not drinking enough fluids, race and ethnicity, age, gender, chronic bladder infections and bladder birth defects.

As used herein “low grade UC” refers to non-muscle invasive tumors at stages below T1, including PUNLMP (see above) and excluding Tis.

As used herein “high grade UC” refers to a UC that is invasive and penetrating to the lamina propria and optionally to the muscle layer.

As used herein “diagnosis” or “diagnosing” refers to determining presence or absence of a pathology (e.g., a disease, disorder, condition or syndrome) in this case, UC, classifying a pathology or a symptom, determining a severity of the pathology (e.g. grade or stage), monitoring pathology progression, forecasting an outcome of a pathology and/or prospects of recovery and screening of a subject for a specific disease.

As mentioned, the methods of some embodiments of the present invention comprise detecting expression of CD24 in a urine sample of the subject.

The urine sample can be obtained using methods known in the art including urine collection or a catheter.

Typically a urine sample obtained by urine collection or a catheter comprises intact cells, shedded cells, exosomes and non-cellular particles.

According to specific embodiments, the urine sample is processed such that the urine sample comprises intact cells in an amount not exceeding 10 cells/ml.

According to specific embodiments, the urine sample is processed such that the urine sample comprises exosomes or non-cellular particles in an amount not exceeding 5×10⁷/ml.

According to specific embodiments, the urine sample is processed such that the urine sample comprises 5×10⁴/ml-5×10⁷/ml, 5×10⁵/ml-5×10⁷/ml or 5×10⁶/ml-5×10⁷/ml exosomes or non-cellular particles.

Thus, according to an aspect of the present invention there is provided a method of detecting CD24 is a subject, the method comprising:

(a) obtaining a urine sample from the subject;

(b) processing said urine sample so as to obtain a urine sample comprising exosomes or non-cellular particles in an amount not exceeding 5×10⁷/ml;

(c) detecting expression of CD24 in said processed urine sample.

Methods of processing urine sample are well known in the art and include, but not limited to, centrifugation.

According to specific embodiments, the processing comprises ultracentrifugation.

According to specific embodiments, the processing does not comprise ultracentrifugation.

According to specific embodiments, the processing comprises centrifugation under a centrifugal force not exceeding 2000×g.

According to specific embodiments, the urine sample used is less than 10 hours, less than 9 hours, less than 8 hours, less than 7 hours, less than 6 hours, less than 5 hours or less than 4 hours.

According to specific embodiments, the urine sample use is less than 6 hours.

Detecting expression of CD24 is effected in-vitro or ex-vivo.

According to specific embodiments, expression of CD24 is detected as a single marker in the method disclosed herein.

According to other specific embodiments, expression of CD24 is detected as part of a signature for bladder cancer not exceeding 3, 2 or 1 additional marker.

That is, according to specific embodiments, the method comprises detecting expression of markers distinct from said CD24, wherein said markers do not exceed 3 distinct markers.

According to specific embodiments, the method comprises detecting expression of markers distinct from said CD24, wherein said markers do not exceed 2 distinct markers.

According to specific embodiments, the method comprises detecting expression of a marker distinct from said CD24, wherein said marker does not exceed 1 marker. As used herein the term “CD24” refers to at least a minimal nucleic acid sequence and/or amino acid sequence that is specific for the phosphatidylinositol-anchored mucin-like cell-surface protein encoded by the CD24 gene (Gene ID: 100133941). According to specific embodiments, CD24 comprises CD24 polypeptide. An exemplary CD24 protein is provided in GenBank Accession No. NP_037362. According to specific embodiments, CD24 comprises CD24 mRNA. An exemplary CD24 transcript is provided in GenBank Accession No. NM_013230.

According to specific embodiments, the CD24 is anchored to intact cells.

According to specific embodiments, the CD24 is not anchored to intact cells. The CD24 not anchored to intact cells can be in a soluble or non-soluble form (e.g. membrane anchored through a GPI moiety). Examples of such CD24 molecule include, but are not limited to secreted CD24 (e.g., CD24 splice variant), shedded CD24 which is devoid of membrane components (e.g., by the action of phospholipases such as PIPLC), blebbed CD24 (i.e., CD24 present in exosomes or non-cellular particles that are formed by rupture of the plasma membrane from the underlying cytoskeleton followed by inflation of the detached membrane by intracellular fluid).

According to specific embodiments, the CD24 is comprised in exosomes or non-cellular particles.

According to specific embodiments, the CD24 is soluble CD24.

As used herein the phrase “predetermined threshold” refers to a level of CD24 that characterizes a healthy urine sample under the same conditions. Such a level can be experimentally determined by comparing samples with normal levels of CD24 (e.g., samples obtained from a healthy subject, e.g., not having cancer) to samples derived from subjects diagnosed with high grade bladder cancer. Alternatively, such a level can be obtained from the scientific literature and from databases.

According to specific embodiments, the predetermined threshold is derived from a control sample.

Several control samples can be used with specific embodiments of the present invention. Typically, the control sample contains a level of CD24 comparable to a healthy urine sample.

Since biological characteristics depend on, amongst other things, species and age, it is preferable that the control sample is obtained from a subject of the same species, age, gender and from the same sub-population.

According to specific embodiments, the control sample is a healthy control sample.

According to specific embodiments, the control sample is a sample from a subject not having a malignancy of the bladder.

According to specific embodiments, the control samples is a sample from a subject having a low grade bladder cancer.

According to specific embodiments, the control sample is obtained from the scientific literature or from a database, such as the known age matched mean value in a non-cancerous population.

According to specific embodiments, the increase above a predetermined threshold is statistically significant.

According to specific embodiments, the predetermined threshold is at least 1.2 fold, at least 1.3 fold, at least 1.4 fold, at least 1.5 fold, at least 1.6 fold, at least 1.7 fold, at least 1.8 fold, at least 1.9 fold, or at least 2 fold as compared the level of CD24 in a control sample as measured using the same assay such as any RNA (e.g. PCR, Northern blot) and/or protein (e.g. ELISA, western blot, flow cytometry) assay suitable for measuring expression level of CD24, as further disclosed herein.

According to specific embodiments, the predetermined threshold is at least 1.5 fold as compared the level of CD24 in a control sample.

Determining the level of CD24 can be affected by any method known in the art, such as but not limited to PCR, RNA chip, Western blot, ELISA, flow cytometry.

As used herein, the phrase “level” when relating to CD24 refers to the degree of gene expression (e.g. mRNA or protein).

It should be noted that the expression level can be determined in arbitrary absolute units, or in normalized units (relative to known expression levels of a control sample). For example, when using RNA chips, the expression levels are normalized according to internal controls or by using quantile normalization.

Expression level can be determined in the urine sample using any structural, biological or biochemical method which is known in the art for detecting the expression level at the transcript or the protein level.

According to specific embodiments, the RNA or the protein molecules are extracted from the urine sample of the subject. Thus, according to specific embodiments, the method further comprises extracting RNA or a protein from the urine sample prior to the detecting.

Methods of extracting RNA or protein molecules from urine samples are well known in the art. The extracted RNA can be further processed to a cDNA. Methods of and commercially available kits for converting RNA to cDNA are well known in the art and include e.g. the use of the enzyme reverse transcriptase. Once obtained, the RNA, cDNA or protein molecules can be characterized for the level of various RNA, cDNA and/or protein molecules using methods known in the arts.

Thus, according to some embodiments, detection of the level of CD24 is performed by contacting the urine sample or fractions or extracts thereof with an agent capable of detecting CD24.

According to specific embodiments, the contacting is effected under conditions which allow the formation of a complex comprising CD24 present in the sample and the agent.

Thus, according to an aspect of the present invention there is provided a composition of matter comprising a urine sample of a subject diagnosed with bladder cancer, and an agent capable of detecting CD24.

According to an additional or an alternative aspect of the present invention there is provided an article of manufacture comprising a urine sample of a subject diagnosed with bladder cancer, and in a separate container an agent capable of detecting CD24.

According to specific embodiments, the urine sample is of a subject having high grade bladder cancer.

According to other specific embodiments, the urine sample is of a subject having low grade bladder cancer.

According to specific embodiments, the composition or the article of manufacture does not comprise an agent capable of detecting markers other than CD24.

According to other specific embodiments, the composition or the article of manufacture comprises agents capable of detecting markers distinct from CD24, wherein said markers do not exceed 3 distinct markers.

According to other specific embodiments, the composition or the article of manufacture comprises agents capable of detecting markers distinct from CD24, wherein said markers do not exceed 2 distinct markers.

According to other specific embodiments, the composition or the article of manufacture comprises agents capable of detecting an additional marker distinct from CD24, wherein said marker does not exceed 1 marker.

According to specific embodiment, detecting the expression level of CD24 is effected at the transcript level using RNA or DNA detection methods.

Thus, according to some embodiments, detection of the level of CD24 is performed by contacting the urine sample or fractions or extracts thereof with an oligonucleotide (e.g. oligonucleotide probe or primer) which specifically hybridizes to a CD24 polynucleotide. Such an oligonucleotide can be at any size, such as a short polynucleotide (e.g., of 15-200 bases), an intermediate polynucleotide of 100-2000 bases and a long polynucleotide of more than 2000 bases.

The oligonucleotide used by the present invention can be any directly or indirectly labeled RNA molecule [e.g., RNA oligonucleotide (e.g., of 17-50 bases), an in-vitro transcribed RNA molecule], DNA molecule (e.g., oligonucleotide, e.g., 15-50 bases, cDNA molecule, genomic molecule) and/or an analogue thereof [e.g., peptide nucleic acid (PNA)] which is specific to CD24 RNA transcript. According to specific embodiments, the oligonucleotide is bound to a detectable moiety.

Oligonucleotides designed according to the teachings of the present invention can be generated according to any oligonucleotide synthesis method known in the art such as enzymatic synthesis or solid phase synthesis.

According to specific embodiments, the contacting is effected under conditions which allow the formation of a complex comprising CD24 mRNA or cDNA present in the sample and the oligonucleotide. The complex can be formed at a variety of temperatures, salt concentration and pH values which may vary depending on the method and the sample used and those of skills in the art are capable of adjusting the conditions suitable for the formation of each nucleotide/probe complex.

Thus, according to an aspect of the present invention there is provided a composition comprising a urine sample of a subject (or an RNA extracted from a urine sample of a subject) diagnosed with bladder cancer and an oligonucleotide capable of detecting a CD24 polynucleotide.

According to another aspect of the present invention there is provided an article of manufacture comprising a urine sample of a subject (or an RNA extracted from a urine sample of a subject) diagnosed with bladder cancer, and in a separate container an oligonucleotide capable of detecting a CD24 polynucleotide.

According to specific embodiments, the composition or the article of manufacture does not comprise more than 10 oligonucleotides capable of detecting 10 distinct markers. According to specific embodiments, the composition or the article of manufacture does not comprise more than 5 oligonucleotides capable of detecting 5 distinct markers. According to specific embodiments, the composition or the article of manufacture does not comprise more than 4 oligonucleotides capable of detecting 4 distinct markers. According to specific embodiments, the composition or the article of manufacture does not comprise more than 3 oligonucleotides capable of detecting 3 distinct markers. According to specific embodiments, the composition or the article of manufacture does not comprise more than 2 oligonucleotides capable of detecting 2 distinct markers. According to specific embodiments, the composition or the article of manufacture comprises only oligonucleotides capable of detecting CD24. According to specific embodiments, the composition or the article of manufacture comprises a single oligonucleotide.

According to specific embodiments, the composition or the article of manufacture further comprises an RNase inhibitor.

Non-limiting examples of methods of detecting RNA and/or cDNA molecules in a sample include Northern blot analysis, RT-PCR [e.g., a semi-quantitative RT-PCR, quantitative RT-PCR using e.g., the Light Cycler™ (Roche)], RNA in-situ hybridization (using e.g., DNA or RNA probes to hybridize RNA molecules present in the cells or tissue sections), in-situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

As mentioned, according to specific embodiments, detecting expression level of CD24 is effected at the protein level using protein detection methods.

Thus, according to some embodiments, detection of the level of the CD24 protein is performed by contacting the urine sample or fractions or extracts thereof with an antibody which specifically binds to CD24. According to specific embodiments, the contacting is effected under conditions which allow the formation of a complex comprising CD24 present in the sample and the antibody (i.e. immunocomplex).

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Several anti-CD24 antibodies that can be used with specific embodiments of the present invention are commercially available, from e.g. R&D, Merck, BioRad and Invitrogen.

Multiple anti-CD24 clones that can be used with specific embodiments of the present invention are known in the art, including SWA11, SN3, C-20, ML-1, ML-5.

According to specific embodiments, the antibody comprises SWA11.

The immunocomplex can be formed at a variety of temperatures, salt concentration and pH values which may vary depending on the method and the sample used and those of skills in the art are capable of adjusting the conditions suitable for the formation of each immunocomplex.

Thus, according to an aspect of the present invention, there is provided a composition comprising a urine sample of a subject (or a lysate of a biological sample of a subject) diagnosed with bladder cancer, and an antibody capable of detecting CD24.

According to an aspect of the present invention there is provided an article of manufacture comprising a urine sample of a subject (or a lysate of a biological sample of a subject) diagnosed with bladder cancer, and in a separate container an antibody capable of detecting CD24.

According to specific embodiments, the composition or the article of manufacture does not comprise an antibody specific for a marker distinct from CD24.

According to specific embodiments, the composition or the article of manufacture comprises an antibody specific for markers distinct from CD24, wherein said markers do not exceed 3 distinct markers.

According to specific embodiments, the composition or the article of manufacture comprises an antibody specific for markers distinct from CD24, wherein said other markers do not exceed 2 distinct markers.

According to specific embodiments, the composition or the article of manufacture comprises an antibody specific for a marker distinct from CD24, wherein said marker does not exceed 1 marker.

According to a specific embodiment, the composition or the article of manufacture further comprises a secondary antibody capable of binding the antibody.

According to specific embodiments, the composition further comprises a protease inhibitor.

Various methods can be used to detect the formation of the CD24 immunocomplex of some embodiments of the present invention well known to those of skills in the art.

According to specific embodiments, detecting the level of expression of CD24 is effected by using an immunoassay using an antibody.

Non-limiting examples of methods of detecting the level of specific protein molecules in a sample include Enzyme linked immunosorbent assay (ELISA), Western blot analysis, immunoprecipitation (IP), radio-immunoassay (RIA), Fluorescence activated cell sorting (FACS) and molecular weight-based approach.

According to a specific embodiment, the immunoassay comprises an ELISA assay.

For Western blot the proteins are extracted from a cell sample and are subjected to electrophoresis (e.g., SDS-PAGE) and blotting to a membrane (e.g., nylon or PVDF). The membrane is then interacted with a CD24 antibody which can be either directly labeled or further subjected to a secondary labeled antibody. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

For immunoprecipitation analysis the CD24 antibody may directly interact with a sample including CD24 and the formed complex can be further detected using a secondary antibody conjugated to beads (e.g., if the CD24 antibody is a mouse monoclonal antibody, the secondary antibody may be an anti-mouse antibody conjugated to e.g., Sepharose beads). The beads can be then precipitated by centrifugation, following which the precipitated proteins (e.g., CD24 and anti CD24 antibodies) can be detached from the beads (e.g., using denaturation at 95° C.) and further subjected to Western blot analysis using the CD24 specific antibodies. Alternatively, the anti-CD24 antibody and the beads-conjugated secondary antibody may be added to the biological sample containing the antigen (CD24) to thereby form an immunocomplex. Alternatively, since CD24 is a highly glycosylated protein, it can be also precipitated using a substrate capable of binding glycosylated polypeptides such Concavalin A (GE Healthcare Bio-Sciences, Uppsala, Sweden) which may be also conjugated to beads, followed by Western blot analysis with anti-CD24 antibodies.

FACS analysis enables the detection of antigens present on cell membranes such as CD24. Briefly, CD24 specific antibodies are linked to fluorophores and detection is performed by means of a cell sorting machine which reads the wavelength of light emitted from each cell as it passes through a light beam. This method may employ two or more antibodies simultaneously.

The expression level of CD24 can be also determined using ELISA. Briefly, a sample containing CD24 antigen is fixed to a surface such as a well of a microtiter plate. An antigen specific antibody (a CD24 antibody) coupled to an enzyme is applied and allowed to bind to the antigen. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

The expression level of CD24 can be also determined using radio-immunoassay (RIA). In one version, this method involves precipitation of the desired antigen (CD24) with a specific antibody and radiolabeled antibody binding protein (e.g., protein A labeled with I¹²⁵) immobilized on a precipitable carrier such as agarose beads. The number of counts in the precipitated pellet is proportional to the amount of antigen.

In an alternate version of the RIA, a labeled antigen and an unlabelled antibody binding protein are employed. A sample containing an unknown amount of antigen is added in varying amounts. The decrease in precipitated counts from the labeled antigen is proportional to the amount of antigen in the added sample.

The level of CD24 can be also determined using molecular weight-based approach. Since the immunocomplex exhibits a higher molecular weight than its components, methods capable of detecting such a change in the molecular weight can be also employed. For example, the immunocomplex can be detected by a gel retardation assay. Briefly, a non-denaturing acrylamide gel is loaded with samples. A shift in the size (molecular weight) of the protein product as compared with its components is indicative of the presence of an immunocomplex. Such a shift to a higher molecular weight can be viewed using a non-specific protein staining such as silver stain or Commassie blue stain.

The antibody or oligonucleotide used by the present invention can be any directly or indirectly labeled antibody or oligonucleotide. According to specific embodiments, the antibody or oligonucleotide is bound to a detectable moiety. Non-limiting examples of detectable moieties include radioactive isotopes, phosphorescent chemicals, chemiluminescent chemicals, fluorescent chemicals, enzymes, fluorescent polypeptides, a radioactive isotope (such as ^{[125]}iodine) and epitope tags.

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.].

Numerous types of enzymes may be attached to the agent [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

Exemplary identifiable moieties include, but are not limited to green flouorescent protein, alkaline phosphatase, peroxidase, histidine tag, biotin, orange fluorescent protein and strepavidin.

The reagents described hereinabove for detection of immunocomplex formation or hybridization when oligonucleotides are used may be included in a diagnostic kit/article of manufacture preferably along with appropriate instructions for use and labels indicating FDA approval for use in e.g. diagnosing high grade bladder cancer and/or for monitoring efficacy of bladder cancer.

Such a kit can include, for example, at least one container including at least one of the above described diagnostic agents (e.g., a CD24 specific antibody, a CD24 oligonucleotide capable of hybridizing CD24) and an imaging reagent packed in another container (e.g., enzymes, secondary antibodies, buffers, chromogenic substrates, fluorogenic material). The kit may also include appropriate buffers and preservatives for improving the shelf-life of the kit.

As mentioned, according to specific embodiments, an increase in the expression level of CD24 above a predetermined threshold as compared to a control sample is indicative of high grade bladder cancer.

It will be appreciated that the presence of high grade bladder cancer can be further validated using additional assays.

Thus, according to specific embodiments, the methods disclosed herein comprise corroborating the diagnosis using a state of the art technique e.g. a Gold standard assay.

Such methods are known in the art and include, but are not limited to, cystoscopy, TURP and ureteroscopy.

Treatment of bladder cancer is based on the tumor's clinical stage, which is how deep it is thought to have grown into the bladder wall and whether it has spread beyond the bladder. Other factors, such as the size and grade of the tumor and a person's overall health, can also affect treatment options. The skilled artisan is aware of the treatment options and the significance of predicting the outcome of treatment.

When needed, according to the present teachings, treating of UC can be effected using methods which are well known in the art. These include, but are not limited to, surgery (TURBT, cystectomy), intravesical therapy (e.g., BCG), chemotherapy (e.g. Mitomycin C), radiation therapy and immunotherapy. Treatment can include each of these options or combinations thereof (e.g., TURBT, BCG).

After resection of all visible tumors, adjuvant intravesical immunotherapy or chemotherapy can be used. Photodynamic therapy and laser ablation have been evaluated as secondary treatments in specific settings.

Distinguishing between low grade (LG) and high grade (HG) UC is of extreme clinical significance, as this distinction affects the therapeutic approach taken. For LG tumors treatment typically encompasses transurethral tumor resection by endoscopic approach. While in HG there is a need for therapy. In localized bladder cancer (T1—only in the mucosa without involvement muscularis), the treatment may typically be intravesical BCG immunotherapy, and radical cystectomy in case of BCG failure. In localized T2 when there is involvement of bladder muscle radical cystectomy is the preferred option. In metastatic bladder cancer chemotherapy and palliative surgery are indicated.

Hence, it will be appreciated that determining the expression level of CD24 has a variety of applications such as, but not limited individually optimizing a treatment for bladder cancer, determining a therapy for a subject and monitoring treatment in a subject.

Thus, according to an aspect of the present invention, there is provided a method of treating bladder cancer in a subject in need thereof, the method comprising:

(a) obtaining a urine sample from the subject;

(b) detecting in said urine sample of the subject expression of CD24;

(c) diagnosing the subject with high grade bladder cancer when said expression of CD24 above a predetermined threshold as compared to a control sample is detected;

(d) administering to the subject an effective amount of a high grade bladder cancer therapy.

According to an additional or an alternative aspect of the present invention, there is provided a method of monitoring treatment of high grade bladder cancer, the method comprising:

(a) treating a subject having high grade bladder cancer with a high grade bladder cancer therapy;

(b) detecting in said urine sample of the subject expression of CD24, wherein a decrease in said expression of CD24 below a predetermined threshold as compared to expression of same prior to treatment is indicative of said therapy being efficacious.

Thus, a decrease in the level of CD24 is indicative of the cancer therapy being efficient. On the other hand, if there is no change in the level of CD24, or in case there is an increase in the level of CD24, then the cancer therapy is not efficient in treating the cancer and additional and/or alternative therapies (e.g., treatment regimens) may be used.

According to specific embodiments of the monitoring aspects disclosed herein, the predetermined threshold is in comparison to the level in the subject prior to cancer therapy.

According to specific embodiments of the monitoring aspects disclosed herein, the predetermined threshold is in comparison to the level in a control sample.

According to other specific embodiments of this aspect of the present invention, the control sample is of a subject having high grade bladder cancer.

According to specific embodiments of the monitoring aspects disclosed herein, the decrease below a predetermined threshold is statistically significant.

According to specific embodiments, the predetermined threshold is at least 1.2 fold, at least 1.3 fold, at least 1.4 fold, at least 1.5 fold, at least 1.6 fold, at least 1.7 fold, at least 1.8 fold, at least 1.9 fold, or at least 2 fold as compared to expression level of CD24 in a control sample or in the subject prior to the cancer therapy as measured using the same assay as any RNA (e.g. PCR, Northern blot) and/or protein (e.g. ELISA, western blot, flow cytometry) assay suitable for measuring expression level of CD24, as further disclosed herein.

According to specific embodiments, the predetermined threshold is at least 1.5 fold as compared the level of CD24 in a control sample or in the subject prior to the cancer therapy.

According to other specific embodiments of this aspect of the present invention, the predetermined threshold can be determined in a subset of subjects with known outcome of cancer therapy. As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Subjects

Urine samples were obtained from volunteers giving their consent. The Institutional Review Board (IRB) of the Tel Aviv Sourasky Medical Center and the Israeli Ministry of Health approved this on patients diagnosed and classified in Tel-Aviv Sourasky Medical Center as having bladder cancer (high or low grade) and underwent tumor resection by endoscopic approach through the urethra into the bladder (TURBT). The obtained urine samples were immediately transferred to the laboratory. Following, the urine samples were centrifuged at 4000 rpm, 4° C. for 5 minutes and the supernatant was kept in 80° C. until testing. All tissue specimens obtained at the time of surgery were sent to a central pathology laboratory. Subjects were thereafter classified based on the histologic findings as: normal (healthy subjects or urologic patients without malignancy of the bladder), TaLG (low grade bladder cancer) or HG (high grade bladder cancer). Tumor size was determined macroscopically.

Sandwich Elisa

ELISA plates were coated with 10 μg/ml capture antibody (humanized anti-CD24 mAb) in PBS overnight at 4° C. All subsequent steps were done at room temperature. The plates were blocked with 3% skim-milk in PBS for 1-2 hours at room temperature. Following incubation, the plates were washed ×3 with PBS. Urine samples were then added and the plates were incubated at 37° C. for 90 min. All subsequent steps were done at room temperature. Following incubation, the plates were washed ×3 with PBS. 10 μg/ml of detection antibody (C-20 goat polyclonal IgG, epitope mapping at the C-terminus of CD24 of human origin, Santa Cruz) diluted in PBS was added and incubated for 2 hours. Following incubation, the plates were washed ×3 with PBS. 100 μl of HRP-conjugated donkey anti-goat (1:5,000 dilution in PBS) was used for detection. Following incubation, the plates were washed ×3 with PBS and the ELISA was developed using the chromogenic HRP substrate TMB. Color development was terminated with 1 M H₂SO₄ and the plates were read at 450 nm.

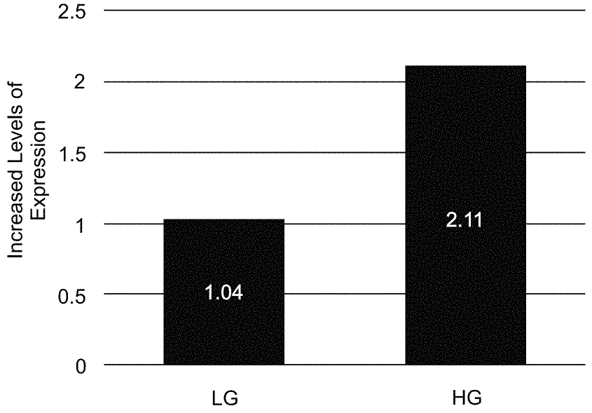

The amount of CD24 in the urine samples was estimated and calculated based on a standard curve (FIGS. 1 and 3) using several concentrations of CD24 purified protein [full length protein. CD24 gene was cloned into pcDNA4/TO plasmid and was stably transfected into the T-REx™ stable cells expressing the tetracycline repressor from the pcDNA6/TR vector (Invitrogen). The expression of CD24 was induced by tetracycline and the protein was purified on affinity column].

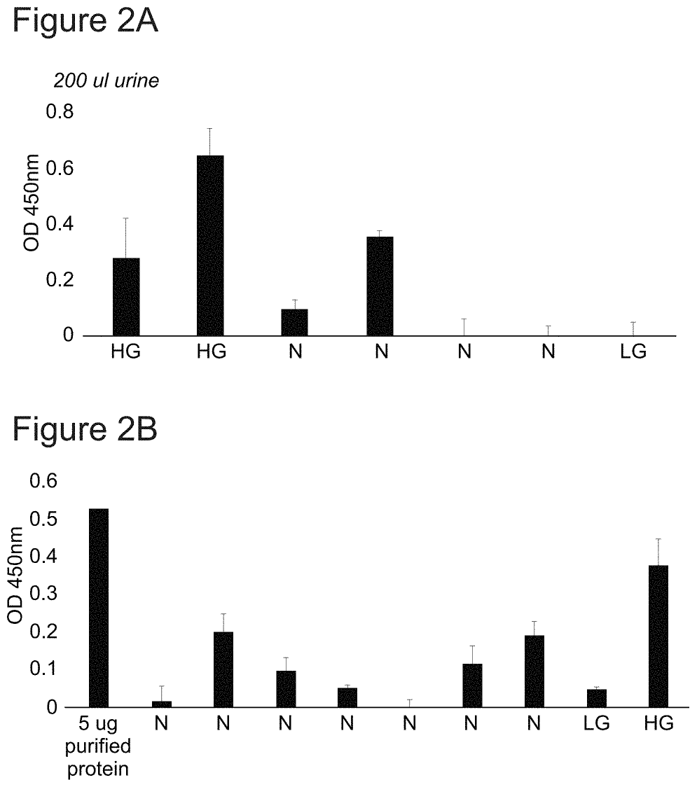

In a similar manner, sandwich ELISA was performed without the reference of a purified protein-based standard curve (FIGS. 2A-B).

### Example 1

### CD24 is Overexpressed in Urine Samples of Patients with High Grade Bladder Cancer

CD24 levels in urine samples of patients diagnosed with bladder cancer were determined using a Sandwich ELISA. The results presented in FIGS. 1, 2A-B and 3 clearly demonstrate that CD24 levels in urine samples obtained from patients having high grade (HG) tumors were higher compared to CD24 levels in urine samples obtained from healthy subjects or urologic patients without malignancy of the bladder; and also compared to CD24 levels in urine samples from patients having low grade (LG) tumors.

The present results suggest that CD24 serves as a new potential and promising biomarker for the detection and surveillance of BC. It is suggested that CD24 is an important prognostic marker for BC which could help to make inroads into improving the outcomes in these patients.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

Choi Y L1, Lee S H, Kwon G Y, Park C K, Han J J, Choi J S, Choi H Y, Kim S H, Shin Y K. Overexpression of CD24: association with invasiveness in urothelial carcinoma of the bladder.
Fillmore C, Kupperwaser C. Human breast cancer stem cell markers CD44 and CD24: enriching for cells with functional properties in mice or in man? Breast Cancer Res 2007; 9:303.
Goodison S, Rosser C J, Urquidi V. Bladder cancer detection and monitoring: assessment of urine- and blood-based marker tests. Mol Diagn Ther 2013; 17:71-84.
Griffiths T R. Action on Bladder Cancer. Current perspectives in bladder cancer management. Int J Clin Pract 2013; 67:435-48.
Jacob J, Bellach J, Grutzmann R, Alldinger I, Pilarski C, Dietel M, and Kristiansen G. Expression of CD24 in Adenocarcinomas of the Pancreas Correlates with Higher Tumor Grades. Pancreatology 2004; 4:454-460.
Kristiansen G, Pilarsky C, Pervan J, Sturzebecher B, Stephan C, Jung K, Loening S, Rosental A, and Dietel K. CD24 Expression Is a Significant Predictor of PSA Relapse and Poor Prognosis in Low Grade or Organ Confined Prostate Cancer. Prostate 2004; 58:183-192.
Kristiansen G, Sammar M, and Altevogt P. Tumor biological aspects of CD24, a mucin-like adhesion molecule. J Mol Histology 2004; 35:255-262.
Kristiansen G, Winzer K J, Mayordomo E, Bellach J, Schluns K, Denkert C, Dahl E, Pilarsky C, Altevogt P, Guski H, and Dietel M. CD24 Expression Is a New Prognostic Marker in Breast Cancer. Clin Can Res 2003; 9:4906-4913.
Lee J H, Kim S H, Lee E S, Kim Y S. CD24 overexpression in cancer development and progression: a meta-analysis. Oncol Rep 2009; 22:1149-56.
Liu C1, Zheng S, Shen H, Xu K, Chen J, Li H, Xu Y, Xu A, Chen B, Kaku H, Nasu Y, Kumon H, Huang P, Watanabe M. Clinical significance of CD24 as a predictor of bladder cancer recurrence. Oncol Lett 2013; 6:96-100.
Overdevest J B, Knubel K H, Duex J E, Thomas S, Nitz M D, Harding M A, Smith S C, Frierson H F, Conaway M, Theodorescu D. CD24 expression is important in male urothelial tumorigenesis and metastasis in mice and is androgen regulated. Proc Natl Acad Sci USA 2012; 109:E3588-96.
Overdevest J B1, Thomas S, Kristiansen G, Hansel D E, Smith S C, Theodorescu D. CD24 offers a therapeutic target for control of bladder cancer metastasis based on a requirement for lung colonization. Cancer Res 2011; 71:3802-11.
Poncet C, Frances V, Gristina R, Scheiner C, Pellissier J F, and Figarella-Branger D. CD24, a glycosylphosphatidylinositol-anchored molecule, is transiently expressed during the development of human central nervous system and is a marker of human neural cell lineage tumors. Acta Neuropathol 1996; 91:400-408.
Rahmani A H, Alzohairy M, Babiker A Y, Khan A A, Aly S M, Rizvi M A. Implication of androgen receptor in urinary bladder cancer: a critical mini review. Int J Mol Epidemiol Genet 2013; 4:150-5.
Sagiv E, Arber N. The novel oncogene CD24 and its arising role in the carcinogenesis of the GI tract: from research to therapy. Expert Rev Gastroenterol Hepatol. 2008; 2:125-33.
Siegel R L, Miller K D, Jemal A. Cancer statistics, 2015. CA Cancer J Clin 2015; 65:5-29.
Smith S C1, Oxford G, Wu Z, Nitz M D, Conaway M, Frierson H F, Hampton G, Theodorescu D. The metastasis-associated gene CD24 is regulated by Ral GTPase and is a mediator of cell proliferation and survival in human cancer. Cancer Res 2006; 66:1917-22.
Ye F, Wang L, Castillo-Martin M, McBride R, Galsky M D, Zhu J, Boffetta P, Zhang D Y, Cordon-Cardo C. Biomarkers for bladder cancer management: present and future. Am J Clin Exp Urol 2014; 2:1-14.

What is claimed is:

1. A method of diagnosing high grade bladder cancer, the method comprising detecting in a urine sample of a subject in need thereof expression of CD24, wherein an increase in said expression of CD24 above a predetermined threshold as compared to a control sample is indicative of said high grade bladder cancer.

2. A method of treating bladder cancer in a subject in need thereof, the method comprising:
(a) obtaining a urine sample from the subject;
(b) detecting in said urine sample of the subject expression of CD24;
(c) diagnosing the subject with high grade bladder cancer when said expression of CD24 above a predetermined threshold as compared to a control sample is detected;
(d) administering to the subject an effective amount of a high grade bladder cancer therapy.

3. The method of any one of claims 1-2, wherein said subject is diagnosed with bladder cancer.

4. A method of monitoring treatment of high grade bladder cancer, the method comprising:
(a) treating a subject having high grade bladder cancer with a high grade bladder cancer therapy;
(b) detecting in said urine sample of the subject expression of CD24, wherein a decrease in said expression of CD24 below a predetermined threshold as compared to expression of same prior to treatment is indicative of said therapy being efficacious.

5. A method of detecting CD24 is a subject, the method comprising:
(a) obtaining a urine sample from the subject;
(b) processing said urine sample so as to obtain a urine sample comprising exosomes or non-cellular particles in an amount not exceeding 5×10⁷/ml;
(c) detecting expression of CD24 in said processed urine sample.

6. A composition of matter comprising a urine sample of a subject diagnosed with bladder cancer, and an agent capable of detecting CD24.

7. The composition of claim 6, wherein said bladder cancer is high grade bladder cancer.

8. The method or the composition of any one of claims 1-7, wherein said urine sample comprises exosomes or non-cellular particles in an amount not exceeding 5×10⁷/ml.

9. The method or the composition of any one of claims 1-8, wherein said urine sample comprises intact cells in an amount not exceeding 10 cells/ml.

10. The method or the composition of any one of claims 1-9, wherein said urine sample is less than 6 hours.

11. The method or the composition of any one of claims 1-10, wherein said CD24 comprises CD24 polypeptide.

12. The method or the composition of any one of claims 1-10, wherein said CD24 comprises CD24 mRNA.

13. The method of any one of claims 1-5 and 8-11, wherein said detecting is by using an immunoassay using an antibody.

14. The method of claim 13, wherein said immunoassay comprises an ELISA assay.

15. The composition of any one of claims 6-11, wherein said agent is an antibody.

16. The composition of claim 15, comprising a secondary antibody capable of binding said antibody.

17. The method or the composition of any one of claims 13-16, wherein said antibody comprises SWA11.

18. The composition of any one of claims 6-10 and 12, wherein said agent is an oligonucleotide.

19. The method of any one of claims 1-4, 8-14 and 17, further comprising corroborating said diagnosis using a Gold standard assay.

20. The method of claim 19, wherein said Gold standard assay is selected from the group consisting of cystoscopy, TURP and ureteroscopy.

21. The method of any one of claims 1-4, 8-14, 17 and 19-20, wherein said control sample is of a healthy subject, a subject not having a malignancy of the bladder or a subject having a low grade bladder cancer.

22. The method of any one of claims 1-4, 8-14, 17 and 19-20, wherein said control sample is of a healthy subject or a subject having a low grade bladder cancer.

23. The method of any one of claims 5, 9-14, 17 and 19-22, wherein said processing comprises centrifugation under a centrifugal force not exceeding 2000×g.

24. The method of any one of claims 5, 9-14, 17 and 19-23, wherein said processing does not comprise ultracentrifugation.

25. The method of any one of claims 1-5, 8-14, 17 and 19-24, wherein said expression of said CD24 is detected as a single marker.

26. The method of any one of claims 1-5, 8-14, 17 and 19-24, wherein said method comprises detecting expression of markers distinct from said CD24, wherein said markers do not exceed 3 distinct markers.

27. The composition of any one of claims 6-12 and 15-18, wherein said composition does not comprise an agent capable of detecting markers distinct from said CD24.

28. The composition of any one of claims 6-12 and 15-18, wherein said composition comprises an agent capable of detecting markers distinct from said CD24, wherein said markers do not exceed 3 distinct markers.
